(19) United States
(12) Patent Application Publication
(10) Pub. No.: US 2024/0415700 A1
(43) Pub. Date: Dec. 19, 2024

(54) Water Ear Guard

(71) Applicant: Khalifah Glover

(72) Inventor: Khalifah Glover

(21) Appl. No.: 18/335,048

(22) Filed: Jun. 14, 2023

(51) Int. Cl. A61F 11/12

(52) U.S. Cl. CPC A61F 11/12

(57) ABSTRACT

A device with a soft rubber ear plug in the shape of an ear canal. A major part of the soft rubber earplug is that it has a disposable clear cap that is attached to the soft rubber gel ear guard that covers and stretches and covers the entire ear lobe securely.

### CROSS-REFERENCE TO RELATED APPLICATIONS

N/A

### STATEMENT REGARDING FEDERALLY SPONSORED RESEARCH OR DEVELOPMENT

N/A

### THE NAMES OF THE PARTIES TO A JOINT RESEARCH AGREEMENT: Khalifah Glover

N/A

### INCORPORATION-BY-REFERENCE OF MATERIAL SUBMITTED AS A TEXT FILE VIA THE OFFICE ELECTRONIC FILING SYSTEM (EFS-WEB)

N/A

### STATEMENT REGARDING PRIOR DISCLOSURES BY THE INVENTOR OR A JOINT INVENTOR

N/A

### BACKGROUND OF THE INVENTION

Water Ear Guard, such as this device, is a product used and handled routinely and, therefore, exposed to water and harsh chemicals. The materials used for the inner ear canal are typically foam that does not have a very highly secure fit or does not protect the inner ear and outer ear. Water ear guard provides a solution to this problem.

### (1) FIELD OF THE INVENTION

The presented application relates to a device that prevents water and harsh chemicals from entering the inner ear or harsh chemicals from harming the outer ear.

### (2) Description of Related Art, Including Information Disclosed Under 37 CFR 1.97 and 1.98

N/A

### BRIEF SUMMARY OF THE INVENTION

By providing a device having an ear canal-shaped soft gel plug to protect the ear canal from water with a clear cap attached that covers the entire ear against resistant protection of harsh chemicals that are acid and base that can cause severe irritation or burns to the skin is reduced. In one embodiment, the lateral view of the structure of the outer ear, pinna, external auditory canal, and ear lobe, and in the second embodiment is the device in its lateral view without being inserted into the inner ear. In another embodiment, the device inserted into the inner ear canal comprises a stretchable attachment to the device being inserted into the ear.

Further features of the device will become apparent from the detailed description.

### BRIEF DESCRIPTION OF THE SEVERAL VIEWS OF THE DRAWING(S)

In the following detailed portion of the present description, the teachings of the present application will be explained in the drawings,

FIG. 1. Is a left lateral view of the structure of an ear according to a first embodiment,

FIG. 2. Is a left lateral view of the structure of a device according to a second embodiment, and

FIG. 3. Is a left lateral view of the structure of a device according to a third embodiment.

### DETAILED DESCRIPTION OF THE INVENTION

In the following detailed description, the device, according to the teachings for this application in the form of a water-ear guard, will be described by the embodiments.

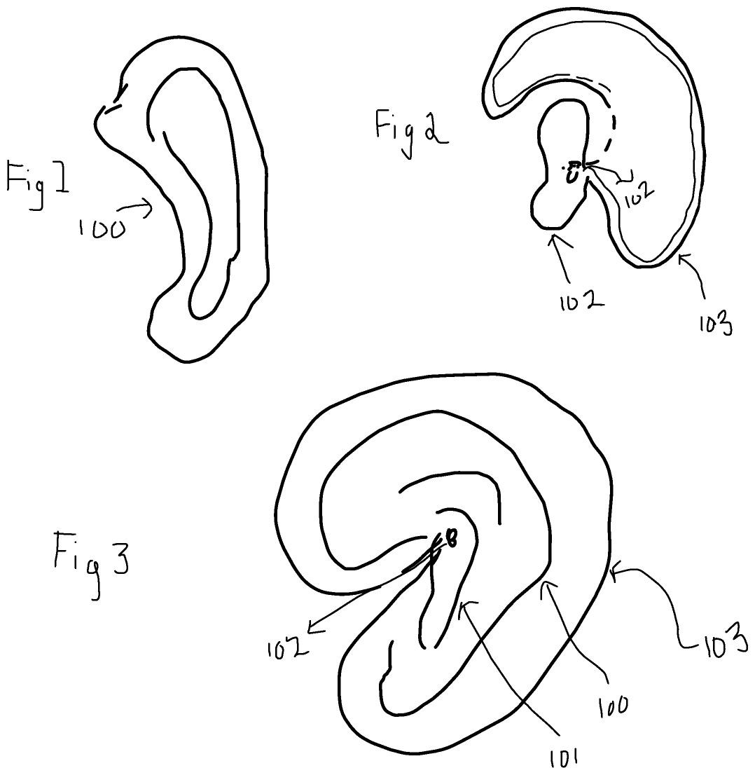

A first embodiment of the structure of ear 100 is illustrated in FIG. 1. The ear 100 comprises a lateral structure of the outer ear, pinna, external auditory canal, and ear lobe. The ear canal-shaped soft rubber gel lateral view 101 comprises an attachment 102, 103, and an attached piece of a clear stretchable cap.

A second embodiment of the water ear guard 101 is illustrated in FIG. 2. The second embodiment is essentially different from the first embodiment, which is a water guard device. The shape of lateral 103 is essentially identical to the shape of the elements of the ear according to the first embodiment.

A third embodiment of the water ear guard attachment 102 is illustrated in FIG. 3. The water ear guard 102, according to the third embodiment, is essentially identical to the ear guard according to the first embodiment, except that the insertion of the ear canal-shaped soft rubber gel lateral view 101, of the front attachment 102, 103 is essentially identical to the shape of the element of according to the first embodiment, a clear stretchable cap wraps around the ear providing protection against water and harsh chemicals.

The invention claimed is:

1. A device comprises an operation of lateral insertion of a device into the external auditory canal, wherein at least a substantial portion of the device has a canal shaped for stabilization. The canal shape device has an attachment piece.

2. A device, according to claim 1, wherein the lateral insertion has a device inserted into the external canal, and a second extent has an attachment piece.

3. A device, according to claim 2, wherein the first extent is a substantial portion of the device that has a canal shaped for stabilization. The attachment piece is a clear stretchable cap that is wrapped behind the pinna of the ear for secureness.
